(19) United States
(12) Patent Application Publication
(10) Pub. No.: US 2026/0365693 A1
(43) Pub. Date: Nov. 19, 2026

(54) Peptide Macrocycles with Embedded Heterocycles

(71) Applicants: The Regents of the University of California, Oakland, CA (US); YALE UNIVERSITY, New Haven, CT (US)

(72) Inventors: Alanna Schepartz Shrader, Berkeley, CA (US); Isaac Knudson, Berkeley, CA (US); Scott J. Miller, New Haven, CT (US); Diondra A. Dilworth, New Haven, CT (US)

(73) Assignees: The Regents of the University of California, Oakland, CA (US); YALE UNIVERSITY, New Haven, CT (US)

(21) Appl. No.: 19/754,164

(22) Filed: Jul. 25, 2026

Related U.S. Application Data
(63) Continuation of application No. PCT/US25/15865, filed on Feb. 13, 2025.
(60) Provisional application No. 63/559,854, filed on Feb. 29, 2024.

(51) Int. Cl. C07K 1/107

(52) U.S. Cl. CPC C07K 1/1077

(57) ABSTRACT

Substituted 2-aminocarbonyl co-substrates are used to provide quinoline-peptide hybrids, or other heterocycle-peptide hybrids, possessing stable and/or conformationally mobile biaryl atropisomeric axes, in methods for the synthesis of peptide macrocycles with embedded heterocycles.

### CROSS-REFERENCES TO RELATED APPLICATIONS

This application is a continuation of PCT/US 25/15865, filed Feb. 13, 2025, which claims priority to U.S. Provisional Application No. 63/559,854, filed Feb. 29, 2024, the disclosures of which are hereby incorporated by reference in its entirety for all purposes.

### GOVERNMENT SUPPORT CLAUSE

This invention was made with government support under grant number 2002182 awarded by the National Science Foundation. The government has certain rights in the invention.

### INTRODUCTION

From the first clinical application of insulin in 19211,2 to the recent triumph of GLP-1 agonists3,4, peptides have proven a valuable therapeutic modality. More recently, advances in genetic code reprogramming, biological display, and computational design have galvanized development of the next generation of peptide-derived therapeutics5. The first wave of these compounds represents some of the most exciting therapeutic candidates of the past decade because of their antibody—like affinity for traditionally “undruggable” targets while simultaneously showing promise for oral or intracellular delivery. Several therapeutic candidates derived from de novo peptide leads have progressed into clinical trials or onto the market6-13. In each of these cases, the initial lead identified by mRNA/cDNA or phage display required extensive modification to improve its physicochemical properties10. Modifications introduced during lead optimization often include the installation of one or more macrocyclic rings and other backbone alterations that enhance protease resistance, target affinity, bioavailability, and/or cell permeability14,15. Chemical strategies to install these modifications earlier in the drug discovery process—ideally, prior to biological display—would offer distinct advantages.

Many strategies exist to install macrocyclic rings within peptides before or after biological display. Common methods include disulfide formation between Cys side chains, thioether formation between a Cys side chain and a terminal chloroacetamide or benzyl halide, copper-catalyzed azide-alkyne cycloaddition, amide bond-forming reactions16-18, and a variety of metal-catalyzed macrocyclizations19-21. Although widely employed, each of these methods suffers from at least one liability with respect to downstream applications and none provide added value. Disulfide bonds are prone to reduction, while thioethers are flexible and, like click reaction products, prone to oxidation. Newer strategies employ nitrile-aminothiol condensations to generate thiazolines22-27 or enzymatic macrocyclizations with tyrosinase28, transglutaminases29, or RiPP enzymes30. Alternative macrocyclization strategies that generate stable and drug-like linkers are widely recognized as an unmet need9,31-34. Chemistry that exploits the macrocyclization event to establish a known pharmacophore within the peptide backbone prior to biological display is a promising strategy towards this end35.

Quinolines are an established class of aromatic pharmacophores replete in natural products, FDA-approved drugs, and high-affinity ligands for both protein and RNA. Quinoline-containing small molecules that engage proteins include FDA-approved therapeutics such as chloroquine, moxifloxacin, topotecan, lenvatinib, and saquinavir, which is itself a peptidomimetic with an N-terminal quinoline36,37. Quinolines are especially privileged with respect to RNA38-41: Quinoline-peptide conjugates function as helix-threading intercalators42,43, and substituted quinolines are found within small molecules that target pre-mRNA to modulate splicing44. Perhaps the most unique aspect of the quinoline pharmacophore is its ability to support axial chirality and exist, in certain cases, as stable atropisomers. This stereochemical complexity would add a new level of topological diversity to genetically encoded or synthetic peptides and could impart long-range conformational control over the peptide structure.

Recently, we reported that linear peptides with a β-dicarbonyl functionality at the N-terminus could be synthesized ribosomally using in vitro translation to generate novel β-dicarbonyl-peptide hybrid molecules45. Other β-dicarbonyl functionality could be introduced into polypeptides using orthogonal aminoacyl-tRNA synthetases46. In both cases, the β-dicarbonyl species reported were derivatives of malonic acid, which are among the least reactive of all 1,3-dicarbonyl species, especially under mild conditions. We hypothesized that if we could ribosomally incorporate a more reactive β-dicarbonyl species at the N-terminus, then the resulting peptide or protein could be modified post-translationally into a heterocyclic pharmacophore using any one of a number of classic or modern chemical transformations. If the post-translational modification made use of a side chain as a co-substrate, then the resulting macrocycle would contain an embedded heterocycle. Such a macrocyclization strategy embodies the post-translational modification logic of RiPP natural products while appropriating chemical intermediates produced by polyketide synthase modules.

### SUMMARY OF THE INVENTION

We disclose that linear peptides with a reactive β- or γ-keto amide at their N-termini can be synthesized ribosomally using in vitro translation methods. Peptides carrying an N-terminal β-keto amide can be converted into diverse quinoline-peptide hybrids via Friedländer reactions with a variety of 2-aminoarylcarbonyl substrates. Reactions with appropriately substituted 2-aminobenzophenones generated quinoline-peptide hybrids with stable biaryl atropisomeric axes. In vitro-translated peptides carrying both an N-terminal β-keto amide and an internal 2-aminoacetophenone motif undergo intramolecular Friedländer macrocyclization reactions that embed a quinoline pharmacophore directly within the macrocyclic backbone. Friedländer macrocyclization reactions proceed on unprotected linear peptides, tolerate proteinogenic functional groups, are mild enough to be employed on in vitro-translated peptides, and do not degrade DNA. When the N-terminal β-keto amide is N-methylated, the Friedländer macrocyclization generates a pair of separable conformational isomers; the structure of one was determined using MicroED. The introduction of N-terminal ketone building blocks into genetically encoded materials and their post-translational derivatization expands the chemical diversity and structural complexity of genetically encoded materials and provides a paradigm for the generation of a wide array of genetically encoded and chemically modified peptides, and the programmed synthesis of peptide-derived materials that more closely resemble complex natural products.

In enumerated aspects and embodiments the invention provides:
1. A method of peptide synthesis comprising:
providing a translation system with a tRNA acylated with a reactive 1,3-dicarbonyl motif under conditions wherein the tRNA introduces the 1,3-dicarbonyl motif as a β- or γ-keto amide at the N-terminus of a nascent peptide, providing a substrate for Friedländer reactions for generating quinoline-peptide hybrid products.
2. The method of claim 1, further comprising converting the N-terminal keto amide into a quinoline-peptide hybrid via a Friedländer reaction with a 2-aminoarylcarbonyl substrate.
3. The method of claim 1, further comprising reacting the N-terminal keto amide with a substituted 2-aminobenzophenone to provide a Friedländer product possessing a stable biaryl atropisomeric axis.
4. The method of claim 1, wherein the peptide comprises the N-terminal keto amide and an internal 2-aminoacetophenone motif, and undergoes an intramolecular Friedländer macrocyclization reaction that embeds a quinoline pharmacophore directly within the
macrocyclic backbone. 6. A method of chemo-ribosomal synthesis of atropisomeric and macrocyclic peptides with embedded heterocycles or pharmacophores, including quinolines, substantially as described herein.
7. A method of ribosomally synthesizing genetically encoded linear peptides with reactive N-terminal β- and γ-ketoamide functionality using in vitro translation.
8. A method herein wherein resultant peptides carrying an N-terminal β-ketoamide provide substrates for Friedländer reactions that convert the β-ketoamide into diverse heterocyclic quinoline-peptide hybrids or heterocycle-peptide hybrids.
9. A method herein wherein use of substituted 2-aminocarbonyl co-substrates provide quinoline-peptide hybrids, or other heterocycle-peptide hybrids, possessing stable and/or conformationally mobile biaryl atropisomeric axes, further expanding stereochemical diversity in genetically encoded peptides, including substituted 2-aminobenzophenone co-substrates that provide quinoline-peptide hybrids.
10. A method herein wherein intramolecular Friedländer reaction of peptides carrying an N-terminal β-ketoamide with an internal 2-aminocarbonyl co-substrate (such as an internal 2-aminobenzophenone co-substrate) or kynurenine residue provide substrates for intramolecular Friedländer cyclization reactions (an effective peptide macrocyclization strategy) that effectively embeds quinoline pharmacophores into the peptide or macrocyclic backbone.

The invention encompasses all combinations of the particular embodiments recited herein, as if each combination had been laboriously recited.

### BRIEF DESCRIPTION OF THE DRAWINGS

FIG. 1A-B. Introduction of a reactive 1,3-dicarbonyl motif at the N-terminus via in vitro translation. a, Scheme illustrating dFx-promoted acylation of tRNA^{fMet} with 3,5-dinitrobenzyl (DNB) esters and use of these tRNAs to initiate the in vitro translation of peptide P1, where X represents N-formyl methionine (fM) or monomers 1-4. DYKDDDDK is a FLAG-tag that enables peptide isolation and enrichment prior to LC-HRMS analysis. b, Yields for flexizyme-promoted acylation of MH and tRNA^{fMet} by the DNB esters of fM and monomers 1-4. Yields were determined using intact tRNA LC-MS46,48.

FIG. 2A-C. Friedländer reactions of β-keto amides proceed readily in aqueous acetic acid. a, Scheme illustrating a classic Friedländer reaction between 2-aminobenzaldehyde and a substituted ketone. b, Model system used to evaluate more mild conditions for the Friedländer reaction of β-keto amide dipeptide 5 and 2-aminoacetophenone 6 (0.33 M each). c, Representative substituted quinoline products generated from Friedländer reactions performed in AcOH at 60 °C for 48 h. Isolated yields are shown in parentheses.

FIG. 3A-F. Post-translational Friedländer reactions on IVT products proceed under mild conditions. a, Post-translational Friedländer reactions of IVT peptide 2-P1 generate diverse quinoline-peptide hybrid molecules denoted Qi-P1, where i identifies the 2-aminocarbonyl substrate. b-e, Reaction scope. Shown is the extracted ion chromatogram (EIC) for the expected major ion produced when 2-P1 is treated under the conditions shown in the presence and absence of substrates 6, 8, 9, or 12 to generate Q6-P1, Q8-P1, Q9-P1, and Q12-P1, respectively. The mass spectrum of each major ion is inset. f, eFx-promoted acylation of tRNA^{fMet} with quinoline cyanomethyl ester 25-CME led to no detectable acylated tRNA product under any condition tested.

FIG. 4A-C. Friedländer reactions of β-keto amides generate stable atropisomers a, Friedlander reaction of substrate 26 and dipeptide 28 in the presence of either Brønsted or Lewis acids generates quinoline-dipeptide 29, which exists as a pair of stable atropisomers with a diastereomeric ratio (d.r.) of ~41: 59 regardless of the conditions used for its preparation. b, X-ray crystal structure of (P, S)-29 (left, CCDC: 2341553) and (M, S)-29 (right, CCDC: 2341552) as their respective hydrochloride salts. c, Friedländer reaction of 2-P1, prepared using IVT, with aminocarbonyl 26 in the presence of 100 mM Yb(OTf)₃ in EtOH generates quinoline-peptide Q26-P1. Extracted ion chromatograms for product formation in the presence of Yb(OTf)₃ in EtOH with 26 (teal), acetic acid with 26 (red), or Yb(OTf)₃ in EtOH without 26 (black) for 24 h at 40 °C. Significantly less product is observed when the reaction is performed using AcOH in place of Yb(OTf)₃ in EtOH.

FIG. 5A-F. Intramolecular post-translational Friedländer macrocyclization reactions. a, Preparative scale intramolecular Friedländer reactions to generate peptide macrocycles with a backbone-embedded quinoline. Each linear peptide contains monomer 2 at the N-terminus and a single internal kynurenine residue. b-e, Structures and total ion chromatograms of crude reaction mixtures containing the Friedländer macrocyclization products CP1-4. The peak containing the cyclic peptide is highlighted in teal. Atom count of the macrocycle backbone, HPLC_{254 nm} yield, and isolated yield (in parentheses) are shown. f, Incorporation of both 2 and kynurenine into a single IVT peptide ivt. LP5 and subsequent Friedländer cyclization into ivt. CP5.

FIG. 6A-B. Aqueous Friedländer macrocyclization of N-methyl β-keto amide peptide LPa generates two distinct isomers. a, N-substitution at the i+1 position in LPa produces stable conformational isomers, CPa1 and (P)-CPa, upon Friedländer cyclization in aqueous sodium phosphate at pH 3.5, favoring the kinetic product CPa1 with a diastereomeric ratio of 76:24. The ostensible atropisomeric axis between the quinoline 3′-carbon and N-methyl amide is highlighted in red. b, MicroED crystal structure (CCDC: 2395208) of (P)-CPa depicting a unique double beta-turn motif. Intramolecular H-bonds shown as dashed orange lines. Cyclic backbone carbon atoms are highlighted in green.

FIG. 7A-B. Synthesis of activated 3,5-dinitrobenzyl (DNB) esters used for microhelix (MH) and tRNA acylation as well as model β-keto amide dipeptide 5 used for Friedländer reaction optimization. a, Schemes illustrating the synthesis of 3,5-dinitrobenzyl (DNB) esters of fMet (fM) and monomers 1-4 used for Flexizyme-promoted acylation of microhelix (MH) and tRNA substrates. b, Scheme showing the synthesis of model β-keto amide dipeptide 5.

FIG. 8. Friedländer reaction mechanism. The Friedländer reaction is a double condensation reaction and can proceed along the paths shown. Precisely which pathway is chosen is dependent on the reaction conditions.7

FIG. 9. Substrates and products of Friedländer reactions.

FIG. 10A-B Additional synthetic routes. Synthetic routes used to prepare the cyanomethyl esters of a, quinoline 25, and b, kynurenine 14.

FIG. 11. Friedländer reaction of model β-keto amide peptide 5 and 2-amino(o-tolyl)phenone (26) to generate 27. Reaction scheme for the generation of atropisomer 27.

FIG. 12. Structures of linear peptides LP1-LP5 containing an N-terminal β-keto amide and an internal kynurenine residue. Linear peptides were prepared using solid phase synthesis, purified, and characterized.

FIG. 13. Cyclic products generated via Friedländer macrocyclization of LP1-5.

### DESCRIPTION OF PARTICULAR EMBODIMENTS OF THE INVENTION

Unless contraindicated or noted otherwise, in these descriptions and throughout this specification, the terms “a” and “an” mean one or more, the term “or” means and/or. It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

### Chemical and Ribosomal Synthesis of Atropisomeric and Macrocyclic Peptides with Embedded Quinolines

In these examples we show that linear peptides with a reactive N-terminal β-keto amide functionality can be synthesized ribosomally using in vitro translation (IVT) and that peptides carrying an N-terminal β-keto amide are substrates for Friedländer reactions capable of generating a diverse set of quinoline-peptide hybrid products. Reaction of the N-terminal β-keto amide with an appropriately substituted 2-aminobenzophenone provides a Friedländer product possessing a stable biaryl atropisomeric axis whose structure could be characterized by NMR and X-ray crystallography. Synthetic or IVT peptides carrying both an N-terminal β-keto amide and an internal kynurenine residue are substrates for intramolecular Friedländer macrocyclization reactions that embed a quinoline pharmacophore into the peptide backbone. When the N-terminal β-keto amide is N-methylated, the Friedländer macrocyclization generates a pair of separable conformational isomers; the structure of one isomer was determined using MicroED. The introduction of simple ketide building blocks into ribosomal synthesis and their post-translational derivatization with carbonyl chemistry expands the structural and stereochemical diversity available to genetically encoded peptides. Overall, this work provides a paradigm for the programmed synthesis of peptide-derived materials that more closely resemble complex natural products.

### Introduction of a Reactive 1,3-dicarbonyl Motif at the N-Terminus Via In Vitro Translation

We began by exploring the extent to which tRNAs could be acylated with monomers carrying a reactive dicarbonyl motif and the extent to which these acylated tRNAs could initiate the in vitro translation of short peptides (FIG. 1a). N-formyl methionine (fM) and monomers 1-4 (FIG. 1b) were converted into activated 3,5-dinitrobenzyl (DNB) esters (FIG. 7a) and their ability to acylate a model tRNA microhelix (MH) in the presence of flexizyme dFx47 was evaluated as a function of time and pH. The extent of MH acylation was monitored using both acidic polyacrylamide gel electrophoresis (aPAGE) and intact tRNA LC-MS46,48. Although gel electrophoresis failed to resolve any acylated MH product from the non-acylated MH substrate, presumably due to similarity in mass and charge, intact tRNA LC-MS revealed that both 2-DNB and 3-DNB were substrates for dFx in MH-acylation reactions, especially at higher pH and longer incubation times. It is likely that the activity of 3-DNB as a substrate for dFx was previously missed because of the low resolving power of gel electrophoresis49. Although we were unable to identify conditions that led to detectable MH acylation by the 3,5-dinitrobenzyl ester of monomer 1 (FIG. 1b), in the case of monomer 4, intact tRNA LC-MS detected formation of acylated MH in 0.6% yield. In vitro-transcribed E. coli initiator tRNA (tRNAf^{fMet} was subsequently acylated on a preparative scale with the 3,5-dinitrobenzyl esters of monomers 2 and 3; in each case, product identity was confirmed using intact tRNA LC-MS.

With acylated tRNA^{fMet} in hand, we next evaluated the extent to which monomers 2 and 3 could be introduced at the N-termini of short peptides prepared using in vitro translation (IVT). We made use of a commercial IVT kit (PURExpress® Δ (aa, tRNA) Kit), supplemented with the requisite amino acids and tRNAs, tRNA^{fMet} pre-acylated with fM or monomers 2 or 3, and a duplex DNA template encoding the FLAG-containing polypeptide MGVDYKDDDDK (MGV-flag). Peptides initiated with monomers 2 or 3 were translated at levels comparable to those initiated with tRNA^{fMet} that had been pre-acylated with N-formyl methionine (fM).

### Friedländer Reactions of β-keto Amides Proceed Readily Under Mild Aqueous Conditions

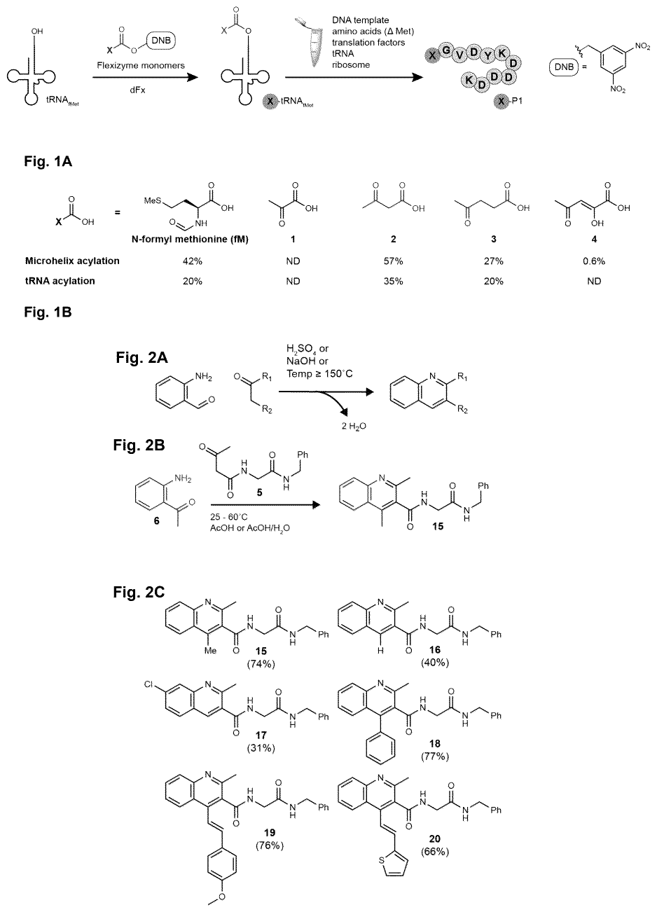

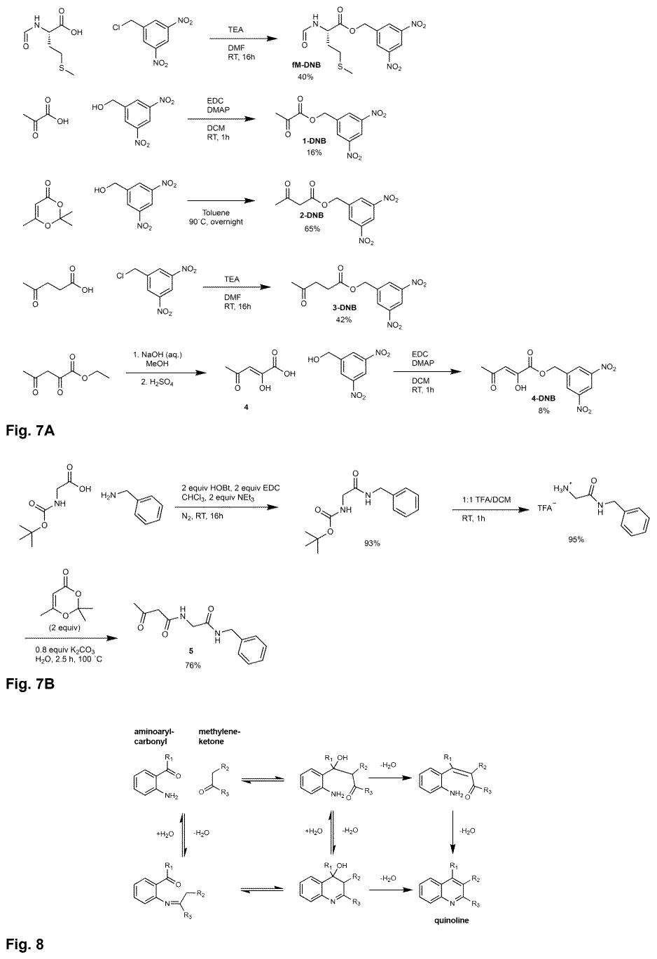

β- and γ-keto carbonyl groups participate in an array of chemical transformations that generate carbocyclic and heterocyclic frameworks, often with elements of stereocontrol50-52. One classic transformation is the Friedländer reaction53, a double condensation of a 2-aminoarylcarbonyl and an aldehyde or ketone to form a quinoline, a fused [6,6] heterocyclic aromatic ring, via the formation of one C—N bond and one C—C bond (FIG. 8). Friedländer reactions are typically performed under harsh conditions, requiring a strongly acidic or basic additive and/or reaction temperatures greater than 150 °C (FIG. 2a)54,55; these conditions would likely degrade sensitive peptides. Although we were encouraged by several reports of high-yielding Friedländer reactions of β-keto esters performed in acetic acid56 and acetic acid/water mixtures⁵⁷, we could identify only limited literature examples of Friedländer reactions of amides⁵⁸⁻⁶¹, and none that proceeded under mild conditions that would be appropriate for peptides and with a wide substrate scope.

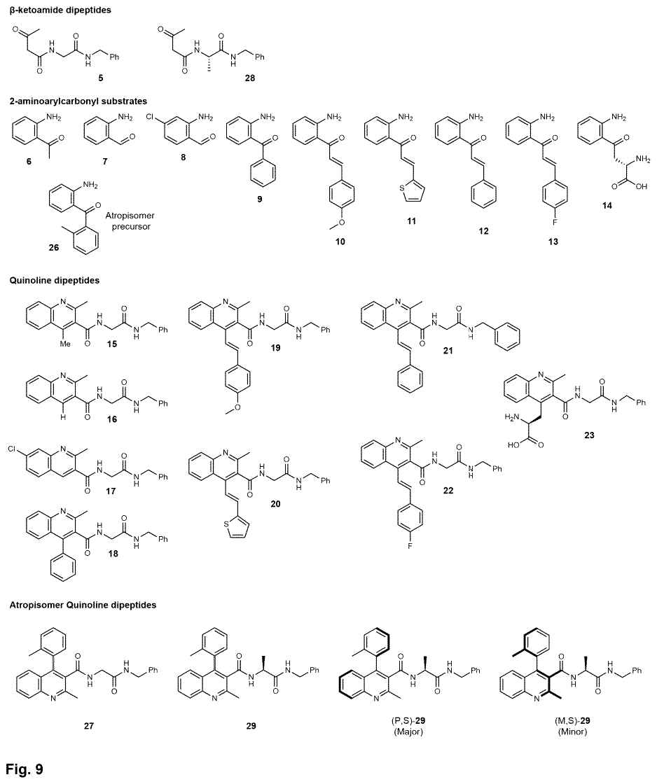

We thus sought to identify mild conditions that would support the Friedländer reaction of model β-keto amide 5 (FIG. 2b), and apply these conditions to peptides generated using IVT. We examined the reaction of 5 with 2-aminoacetophenone 6 to generate quinoline 15 in acetic acid and acetic acid/water mixtures at temperatures between RT and 60 °C. Reactions were performed using 330 mM of 5 and 6 and the formation of quinoline 15 was monitored as a function of time using LC-MS. We found that all conditions tested supported the Friedländer reaction of β-keto amide 5 to generate the expected quinoline product 15. Conversion was most rapid at 60 °C in AcOH or H₂O/AcOH mixtures or at 40 °C in AcOH. To evaluate reaction scope under these conditions, we treated β-keto amide 5 with a collection of nine structurally diverse 2-aminoarylcarbonyl substrates 6-14 (FIG. 2c and FIG. 9). Substrates 6 and 9-13 reacted cleanly with β-keto amide 5 with full conversion to the expected quinoline products 15 and 18-22 after 48 h at 60 °C in AcOH. Reactions with 2-aminobenzaldehydes 7 and 8, which polymerize under acidic conditions62, and kynurenine 14, led to lower levels of product formation. Quinolines 15-20 were synthesized on a preparative scale and the products were characterized using LC-HRMS and NMR.

### Post-Translational Friedländer Reactions of IVT Products Proceed Under Mild Conditions

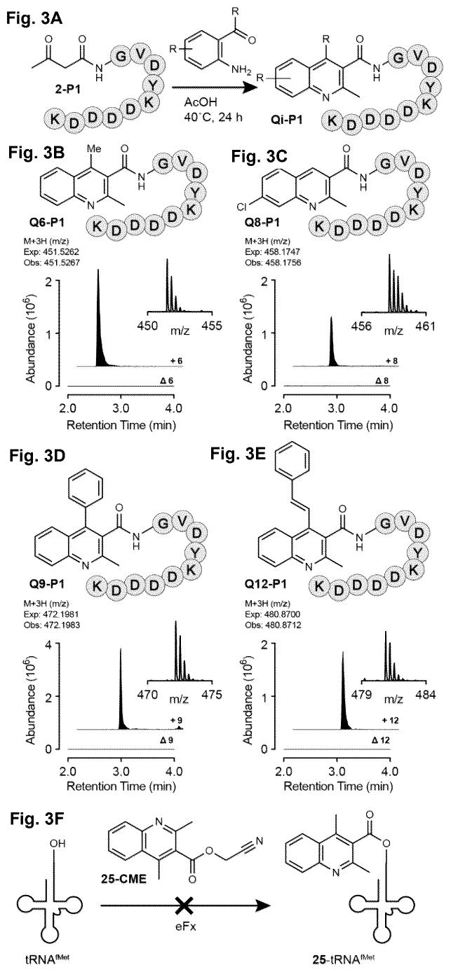

We next asked whether Friedländer reactions would also proceed efficiently when performed on IVT peptides initiated with tRNA^{fMet} acylated with monomer 2. Performing reactions on in vitro translation products is challenging because the reaction must proceed cleanly at low peptide concentration (<1 μM) and in complex reaction mixtures. β-keto amide peptide 2-P1 was generated using IVT, isolated using anti-FLAG magnetic beads, lyophilized, and treated with 2-aminoarylcarbonyl substrates 6, 8, 9, or 12 in acetic acid at 40 °C for 24 h. The formation of Friedländer products Q6-P1, Q8-P1, Q9-P1, and Q12-P1 was monitored using LC-HRMS. In each case, the post-translational Friedländer reaction of 2-P1 proceeded to generate peptides with quinoline derivatives at their N-termini (FIG. 3b-e). In theory, identical quinoline-peptide hybrids could be prepared ribosomally using tRNA^{fMet} acylated with a pre-assembled quinoline. Yet treatment of tRNA^{fMet} with eFx and dimethyl quinoline cyanomethyl ester 25-CME (FIG. 10A-B) led to no detectable acyl-tRNA product under any condition tested (FIG. 3f). We conclude that post-translational Friedländer reactions represent a simple path to ribosomally synthesized peptides with diverse quinoline functionality at the N-terminus.

### Friedländer Reactions of β-keto Amides Generate Stable Atropisomers

Appropriately substituted quinolines can exist as atropisomers. Canonical atropisomers are stereoisomers that arise from hindered rotation around a σ-bond63. Like stereoisomers with point chirality, atropisomers can bind macromolecular targets with distinct kinetics and affinities64. Atropisomeric quinolines and quinoline-like heterocycles are well known. They are found in pharmaceuticals65-67 such as Sotorasib, a first-in-class mutant KRAS G12C inhibitor58,59, as well as novel HIV-I integrase inhibitors70. Atropisomeric quinolines also serve as the molecular framework for catalysts with widespread use in organic synthesis and process research, such as QUINAP65. Although many macrocyclic peptide natural products contain unusual structural isomers, sometimes called atropisomers71, non-canonical atropisomers72, or ansamers73, that result from threaded-loop or knot-like structures72,74,75, true canonical atropisomers are also important constituents of this class of compounds, such as the famous case of the cyclic glycopeptide vancomycin76. Peptides or cyclic peptides containing atropoisomeric quinolines would add a sophisticated layer of stereochemical complexity to macrocyclic peptide libraries.

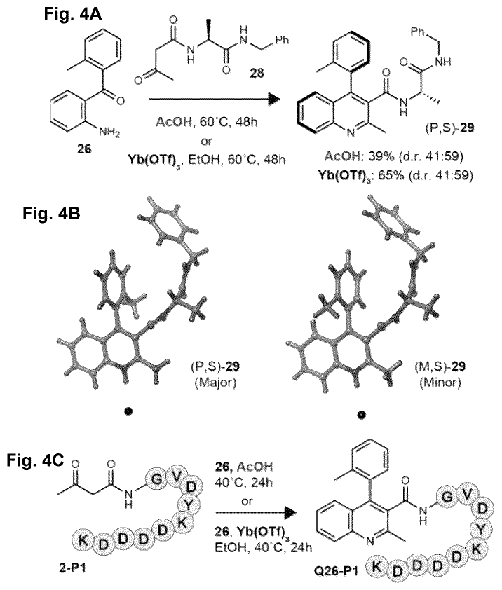

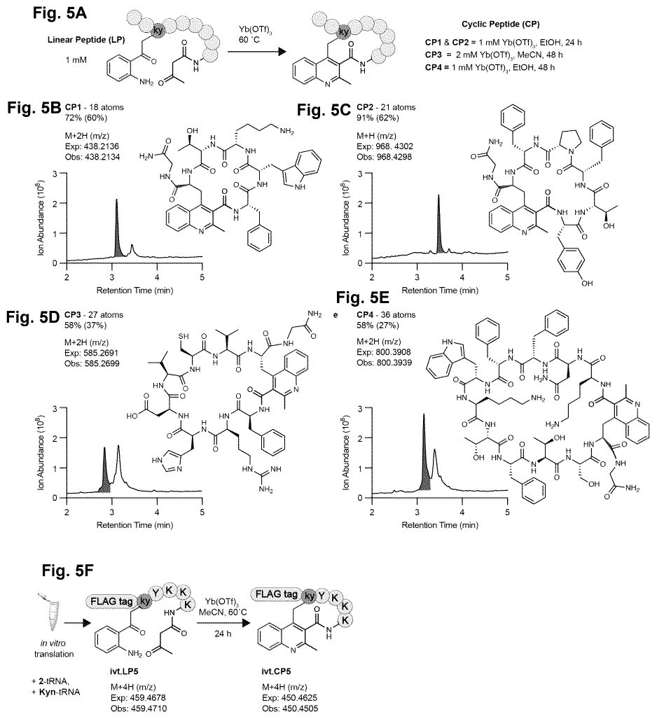

To assess whether atropisomeric quinolines could be introduced within ribosomal peptides, we first confirmed that Friedländer reaction of peptide 28 with (2-aminophenyl)(o-tolyl)methanone (26) in AcOH at 60 °C for 48 h produced the expected product 29 as a roughly 41:59 mixture of atropisomeric products, as determined by ¹H NMR (FIG. 4a). However, when we attempted to reproduce a post-translational Friedländer reaction between (2-aminophenyl)(o-tolyl)methanone (26) and IVT-generated peptide 2-P1 in acetic acid, the expected product Q26-P1 was detected, but in low abundance.

### Lewis Acid Catalysis of Post-Translational Friedländer Reactions Generates Atropisomeric Products

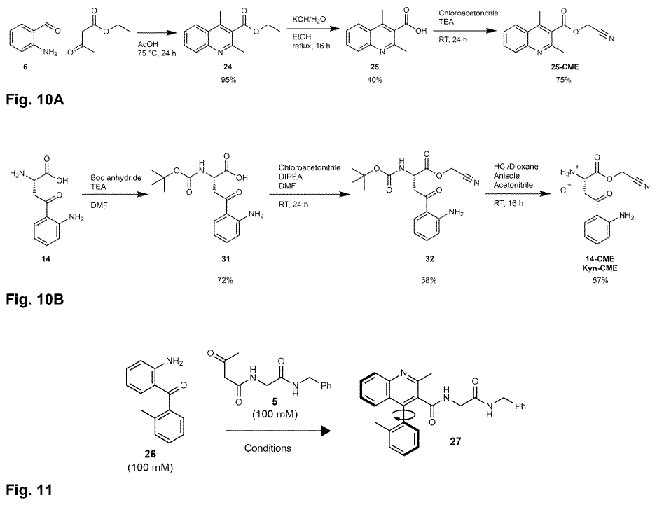

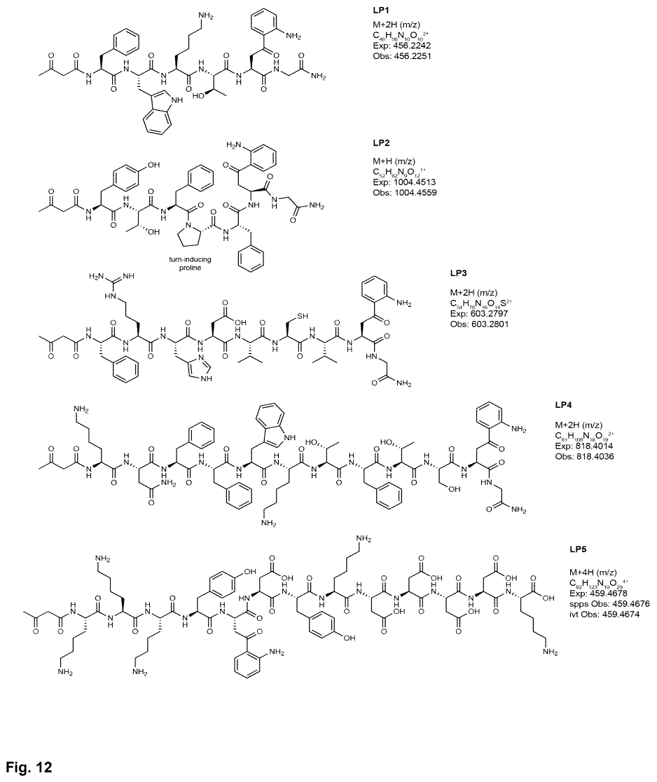

We hypothesized that Friedländer reactions of β-keto amide-containing peptides would proceed under mild Lewis acid catalysis, conditions that would be unlikely to harm ribosomal peptides generated by IVT. Indeed, Lewis acids such as the chloride salts of Ce(III)77 and Fe(III)78 as well as the triflate salts of Yb(III)79, Y(III)80, and Zn(II)81 promote Friedländer reactions of β-keto esters⁵⁵. To evaluate if they could improve atropisomer-forming reactions of β-keto amide of 2-P1, we screened their effect (at 20 mol %) on the Friedländer reaction of dipeptide 5 and (2-aminophenyl)(o-tolyl)methanone (26) to generate the quinoline 27 (FIG. 11). Of all Lewis acids tested, the highest yields were obtained with Yb(OTf)₃ in ethanol, but Yb(OTf)₃ also performed well in DCM, MeCN, THF, and AcOH. Although strong Brønsted acids such as triflic acid, sulfuric acid, phosphoric acid, and trifluoroacetic acid also out-performed acetic acid with regard to the preparation of 27, these conditions are far less mild.

When the Friedländer reaction of 28 with 26 was repeated, this time in the presence of 20 mol % Yb(OTf)₃ in EtOH at 60 °C for 48 h, we isolated quinoline 29 in 65% yield with a similar 41:59 diastereomeric ratio (FIG. 4a). The diastereomers were separated and characterized by ¹H NMR and X-ray structure determination (FIG. 4b), revealing the (P, S) diastereomer as the major product. The stability of the atropisomeric axis was assessed by heating a pure sample of the minor diastereomer (i.e., (M, S)-29) in DMSO-d₆ at 100 °C for 18 h 82 . Subsequent ¹H NMR analysis revealed minimal atropisomerization (<1%). With optimized conditions in hand, we again attempted modification of a ribosomal peptide generated by IVT. Indeed, when treated with 26 in EtOH containing Yb(OTf)₃, the IVT synthesized β-keto amide peptide 2-P1 was transformed cleanly into the quinoline-peptide hybrid Q26-P1 after 24 h at 40 °C (FIG. 4c). Q26-P1 is presumed to exist as a mixture of atropisomers in analogy to 29, but chromatographic separation of distinct isomers by reverse phase UPLC-MS was not observed on this larger IVT peptide.

### Friedländer Macrocyclizations

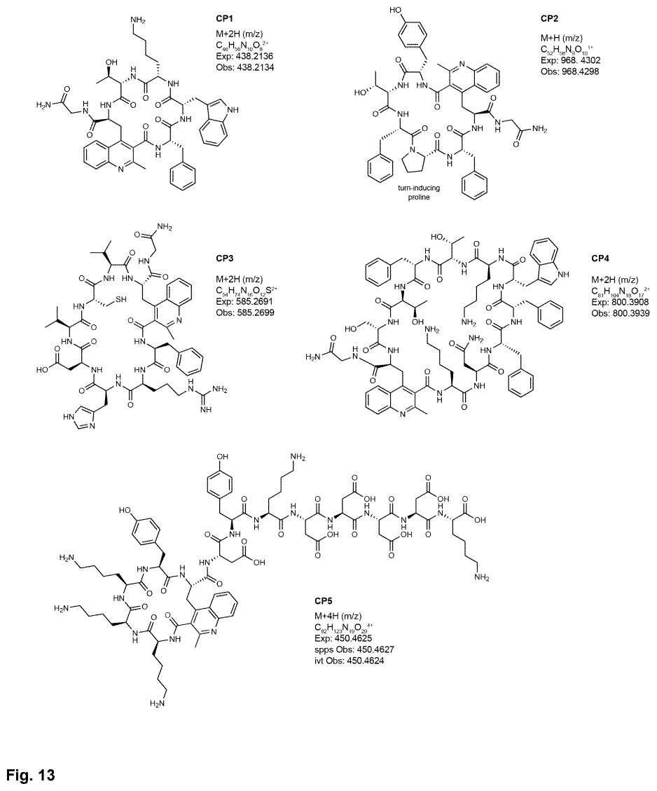

We envisioned that Lewis acids might also aid post-translational Friedländer macrocyclization reactions to generate products with a quinoline pharmacophore embedded within the peptide backbone. To test this idea, we prepared four linear peptides containing both an N-terminal β-keto amide and an internal 2-aminoarylcarbonyl side chain provided by the non-canonical amino acid kynurenine (FIG. 5a and FIG. 12). The linear peptide LP1 is a heptapeptide related to the FDA-approved drug octreotide5,83, while LP2 is a hexapeptide containing a turn-inducing L-proline, LP3 is a decapeptide bearing diverse functional groups, and LP4 is a longer diverse tridecapeptide related to the hormone somatostatin84. All four linear peptides LP1-4 (1 mM) underwent intramolecular Friedländer reactions to generate macrocycles CP1-4 (FIG. 13) in the presence of 1-2 mM Yb(OTf)₃ in EtOH or MeCN at 60 °C, as supported by LC-HRMS (FIG. 5b-e) and NMR experiments). The smaller peptides LP1 and LP2 cyclized cleanly and the macrocyclic products CP1 (72% HPLC and 60% isolated yield) and CP2 (91% HPLC and 62% isolated yield) could be isolated with efficiencies that tracked well with LC-MS and HPLC chromatograms of reaction progress. Cyclization of the larger peptides LP3 and LP4 proceeded under analogous conditions to deliver their corresponding macrocycles with reduced, but still notable efficiency: CP3 (58% HPLC and 37% isolated yield) and CP4 (58% HPLC and 27% isolated yield). LC-HRMS analysis of the crude macrocyclization product revealed residual unreacted linear peptide as well as (in the case of LP3) single-dehydration byproducts, indicating incomplete conversion under the conditions tested. Cyclization of long linear peptides is known to be challenging in the absence of turn-inducing elements85. The presence of single-dehydration byproducts in the reaction of LP3 could result from side reactions, such as aspartimide formation, or indicate the accumulation of Friedländer reaction intermediates. For comparison, macrocyclization reactions of LP1-4 were also performed under aqueous conditions (80 mM glycine pH 2.8 at 60 °C); in all cases, cyclization proceeded faster using the Lewis acid Yb(OTf)₃. The isolated macrocycles CP1-CP4 were also characterized by MALDI-TOF MS to confirm the monomeric mass and the absence of significant quantities of higher order oligomers.

Next, we examined whether Lewis acid-catalyzed Friedländer macrocyclization conditions were compatible with the nucleic acids used to establish genetically encoded libraries. We screened a series of Friedländer reaction conditions to identify those that would support both the cyclization of LP2 and the stability of a 28-mer DNA: RNA duplex. We find that the Yb(OTf)₃-promoted Friedländer macrocyclization of LP2 proceeds well in MeCN containing 20% H₂O, a minimum requirement for oligonucleotide solubility in genetically encoded libraries86. Although Friedländer macrocyclization efficiency is lower in MeCN containing 50% H₂O, it can be rescued using super-stoichiometric Yb(OTf)₃. These conditions result in substantial degradation of the RNA strand, but the DNA remains intact. This observation indicates that Friedländer reactions could be used in the context of either DNA-encoded or mRNA/cDNA-display libraries87,88.

Finally, we asked whether a Lewis acid-catalyzed Friedländer macrocyclization reaction was compatible with the challenging conditions required for post-translational modifications of in vitro translation (IVT) products. First, the cyanomethyl ester of kynurenine (Kyn-CME) and the flexizyme eFx were used to acylate tRNA^{AsnE2#2}_{GCU}, a previously reported engineered tRNA89. The resulting Kyn-tRNA^{AsnE2#2}_{GCU} was added to the IVT reaction while excluding serine and sequestering native tRNA^{Ser}_{GCU} with an antisense oligonucleotide (M5-1) to suppress near cognate read-through22,90. We confirmed that β-keto acid 2 and kynurenine (Kyn) could independently and effectively recode methionine at position 1 and serine at position 6, respectively, of a peptide prepared using IVT. Using an identical strategy, we then used IVT to generate tetradecapeptide ivt. LP5, which contains both an N-terminal β-keto amide as well as an internal kynurenine residue (FIG. 5f). Subsequent reaction of ivt. LP5 with 100 mM Yb(OTf₃) in MeCN at 60 °C for 24 h yielded quinoline-cyclized peptide ivt.CP 5 (FIG. 5f) whose retention time and mass spectrum were identical to that of an authentic standard produced by SPPS. This result confirms that mild, post-translational Lewis acid-catalyzed Friedländer reactions can effectively generate macrocyclic quinoline-peptide hybrids even at very low peptide concentrations and in the presence of the high concentrations of salts and biomolecules that are intrinsic to in vitro translation reactions.

### Atroposelective Friedländer Macrocyclizations

Aryl-tertiary amide atropisomers are well-studied conformational isomers that have garnered significant attention owing to their room-temperature stability and complex stereodynamics91. Yet, these systems have never been employed in peptides. Noting the steric congestion around the quinoline-amide bond in macrocycles CP1-5, we hypothesized that N-methylation of the N-terminal β-keto amide would increase the barrier to rotation about the quinoline-amide bond and lead to isolable diastereomeric quinoline products. If so, the quinoline-amide bond would function as a “dominant rotor” to stabilize a unique peptide conformation92,93.

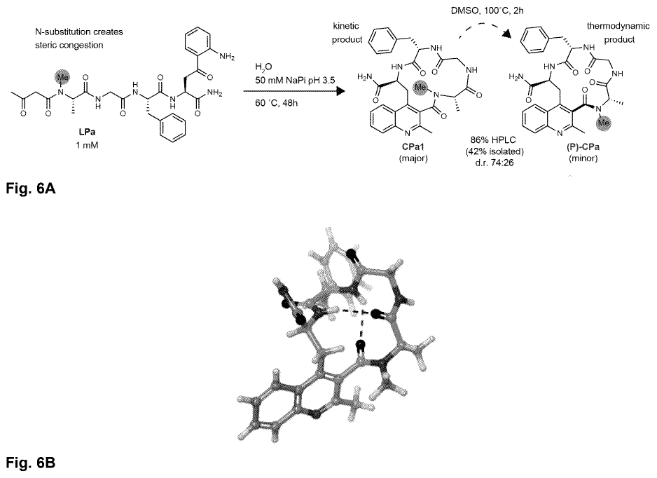

To test this hypothesis, we synthesized peptide LPa, which contains N-methyl-alanine at position 2, and evaluated its macrocyclization under various conditions (FIG. 6a). When heated in aqueous sodium phosphate buffer at pH 3.5, LPa was converted into a pair of isobaric products that were readily separable by reverse phase HPLC. Both bore the characteristic ¹H NMR and absorbance signatures of a quinoline heterocycle. The two products were isolated in 42% yield (86% HPLC conversion) and a ratio of 76:24 (HPLC). The minor isomer formed small needle-like crystals during methanol evaporation and was characterized using MicroED94. The resulting structure (FIG. 6b, CCDC: 2395208), solved at a resolution of 0.9 Å, shows a (P)-configured atropisomer about the quinoline-amide C—C axis, along with trans-amides at each amide linkage. In this structure, the N-methyl group points away from the rest of the peptide macrocycle and the tertiary amide oxygen points inward to accept an H-bond from the i+3 Phe N—H. (P)-CPa adopts a unique double beta-turn motif (type II′ followed by type I), with two H-bonds that cross in the interior of the structure. This type of double turn is rare in proteins, accounting for only 2.2% of double turns seen in the PDB 95. This observation emphasizes the ability for formation of unique macrocyclization motifs in these Friedländer cyclizations; moreover, the installation of an axis of chirality clearly introduces an element of stereogenicity to the conformational landscape of these peptidic structures.

### Discussion

Peptides and their derivatives are a rapidly expanding class of therapeutic molecules, in part because they condense the expansive recognition properties of a protein into a more compact molecular framework. The extended binding surface of a peptide provides a better complement to the extended and featureless surfaces that comprise many traditionally “undruggable” targets96. Perhaps the greatest attribute of peptides, whether linear or cyclic, is that they are genetically encodable, and hence their properties can be optimized using directed evolution methods performed in vitro or in vivo. Many of these methods depend on ribosomal translation and reliably return peptide leads for challenging and clinically relevant targets. For these reasons, de novo peptides have attracted significant attention as the next frontier in pharmaceutical development97.

Yet the difference between the de novo peptide lead and the final compound advanced to clinical trials is often profound, and the modifications introduced demand exceptional commitments of labor and state-of-the-art medicinal chemistry98. Introducing these modifications earlier in the discovery process would be advantageous. Some important progress has been made, including the introduction of N-Me groups99, D-amino acids100, β²- and β³-amino acids, as well as linkers that introduce rings7 or even appended pharmacophores24,35. Even with these developments, the chemistry available to ribosomally translated peptides remains limited when compared to the vast array of transformations, functional groups, and architectures that are emblematic of small molecule pharmaceuticals and bioactive natural products. Yudin and others have shown that the introduction of heterocyclic grafts into peptide macrocycles can have profound impacts on passive permeability, bioavailability, and even topological complexity92,101. Quinolines, however, are underrepresented among these grafts; thus, the properties imbued by these motifs represent an area ripe for further exploration. Well-known properties of quinolines, such as turn-on fluorescence under acidic conditions102,103, oligonucleotide binding38, and atropisomer scaffolding82, are valuable additions to peptide chemistry.

This work was initiated in an attempt to generate ribosomally synthesized peptides with modifications that resemble those found in peptide-derived clinical candidates. We accomplished this goal by installing a reactive β-keto amide functional group at the N-terminus and developing mild Friedländer reaction conditions to convert the β-keto amide into quinoline-peptide hybrids, including those that exist in distinct atropisomeric forms. The same mild Friedländer conditions were deployed as a macrocyclization strategy, embedding a quinoline pharmacophore directly into the backbone of a sequence-encoded macrocycle. Peptides containing N-methyl β-keto amides undergo intramolecular Friedländer reactions to generate separable conformational isomers, providing a new strategy to achieve conformational control and increase the stereochemical diversity of a macrocyclic peptide.

Finally, we note the N-terminal β-keto amide and γ-keto amide peptides described here are potential substrates for many other macrocyclization or late-stage diversification reactions51. Potential reactions of N-terminal β- and γ-keto amides include those that generate pyridines (Hantzsch reaction104), pyrazoles (Knorr105), pyrroles (Hantzsch, Knorr, Paal-Knorr106), dihydropyrimidinones (Biginelli107), and carbocycles (Conia-Ene108), in addition to Michael additions, alkylations, acylations, and arylations. Some of these reactions also have the potential to install atropisomeric axes into the macrocyclic product. These modifications could be used alone or combined with more traditional backbone diversification strategies to generate biased libraries containing pharmacophores and drug-like motifs. We envision the ribosomal incorporation of β- and γ-keto amides as an important development in the late-stage functionalization and macrocyclization of peptides with validated drug-like features. The simplicity of the substrates, tolerance of diverse functional groups, and mildness of the chemistry indicate that Friedländer reactions find utility beyond peptides, in protein functionalization or macromolecule bioconjugation.

### REFERENCES

1. Sims, E. K., et al. Nat. Med. 27, 1154-1164 (2021).
2. Banting, F. G., et al. Can. Med. Assoc. J. 12, 141-146 (1922).
3. Wilding, J. P. H. et al. N. Engl. J. Med. 384, 989-1002 (2021).
4. Müller, T. D., et al. Nat. Rev. Drug Discov. 21, 201-223 (2022).
5. Muttenthaler, M., et al. Nat. Rev. Drug Discov. 20, 309-325 (2021).
6. Costa, L., et al. Pharmaceuticals 16, 996(2023 ).
7. Zorzi, A. et al. Curr. Opin. Chem. Biol. 38, 24-29 (2017).
8. Yang, J. et al. 12, (2022).
9. Ohta, A. et al. J. Am. Chem. Soc. (2023) doi: 10.1021/jacs.3c07145.
10. Tucker, T. J. et al. J. Med. Chem. 64, 16770-16800 (2021).
11. Li, X., et al. J. Med. Chem. 65, 11913-11926 (2022).
12. Sawyer, T. inContemporary Accounts in Drug Discovery and Development(eds. Huang, X., Aslanian, R. G. & Tang, W. H.) 273-292 (Wiley, 2022).
13. Howard, J. F. et al. Lancet Neurol. 22, 395-406 (2023).
14. Rezai, T., et al. J. Am. Chem. Soc. 128, 2510-2511 (2006).
15. Rossi Sebastiano, M. et al. J. Med. Chem. 61, 4189-4202 (2018).
16. He, J., et al. Chem. Sci. 15, 2300-2322 (2024).
17. White, C. J. et al. Nat. Chem. 3, 509-524 (2011).
18. Bechtler, C. et al. RSC Med. Chem. 12, 1325-1351 (2021).
19. Miller, S. J., et al. J. Am. Chem. Soc. 118, 9606-9614 (1996).
20. Walensky, L. D. et al. J. Med. Chem. 57, 6275-6288 (2014).
21. Rivera, D. G., et al. Chem. Soc. Rev. 49, 2039-2059 (2020).
22. Liu, M. et al. Chem. Sci. (2023) doi: 10.1039/D3SC03117A.
23. Liu, M. et al. Chem.-Eur. J. n/a,.
24. Iskandar, S. E. J. Am. Chem. Soc. 145, 1512-1517 (2023).
25. Abdelkader, E. H. et al. Angew. Chem. Int. Ed. 61, e202114154(2022 ).
26. Patil, N. A. et al. ACS Med. Chem. Lett. 12, 732-737 (2021).
27. Thombare, V. J. et al. Chem.—Eur. J. 30, e202401674(2024 ).
28. Fleming, M. C., et al. J. Am. Chem. Soc. 145, 10445-10450 (2023).
29. Bowler, M. M., et al. ACS Chem. Biol. 18, 166-175 (2023).
30. Vinogradov, A. A. et al. J. Am. Chem. Soc. 144, 20332-20341 (2022).
31. Passioura, T. Biochemistry 59, 139-145 (2020).
32. Melsen, P. R. A., et al. ChemBioChem 23, e202100685(2022 ).
33. Smolyar, I. V., et al. Chem. Rev. 119, 10032-10240 (2019).
34. Dengler, S. et al. Angew. Chem. Int. Ed. 62, e202308408(2023 ).
35. Ekanayake, A. I. et al. J. Am. Chem. Soc. 143, 5497-5507 (2021).
36. Yadav, P. et al. Bioorganic Chem. 109, 104639(2021 ).
37. Musiol, R. Expert Opin. Drug Discov. 12, 583-597 (2017).
38. Childs-Disney, J. L. et al. Nat. Rev. Drug Discov. 21, 736-762 (2022).
39. Costales, M. G., et al. J. Med. Chem. 63, 8880-8900 (2020).
40. Haniff, H. S., et al. SLAS Discov. 25, 869-894 (2020).
41. Donlic, A. et al. ACS Chem. Biol. 17, 1556-1566 (2022).
42. Krishnamurthy, M., et al. Org. Lett. 6, 63-66 (2004).
43. Krishnamurthy, M., et al. Angew. Chem. Int. Ed. 46, 7044-7047 (2007).
44. Chen, J. L. et al. J. Am. Chem. Soc. 142, 8706-8727 (2020).
45. Ad, O. et al. in Vitro. ACS Cent. Sci. 5, 1289-1294 (2019).
46. Fricke, R. et al. Nat. Chem. 15, 960-971 (2023).
47. Murakami, H., et al. Nat. Methods 3, 357-359 (2006).
48. Fricke, R., et al. https://doi. org/10.1101/2023.07.14.549096(2023 ).
49. Lee, J. et al. Nat. Commun. 13, 6322(2022 ).
50. Li, W., et al. Eur. J. Org. Chem. 2021, 5151-5192 (2021).
51. Wu, X. et al. J. Heterocycl. Chem. 59, 1445-1490 (2022).
52. Amarnath, V. et al. J. Org. Chem. 60, 301-307 (1995).
53. Friedlaender, P. Berichte Dtsch. Chem. Ges. 15, 2572-2575 (1882).
54. Cheng, C.-C. et al. inOrganic Reactions37-201 (John Wiley & Sons, Ltd, 2005).
55. Marco-Contelles, J., et al. Chem. Rev. 109, 2652-71 (2009).
56. Meléndez, A. et al. Synthesis 52, 1804-1822 (2020).
57. Shen, Q. et al. Synthesis 44, 389-392 (2012).
58. Ţînţaş, M.-L. et al. Eur. J. Med. Chem. 155, 171-182 (2018).
59. Atechian, S. et al. Tetrahedron 63, 2811-2823 (2007).
60. Gopi, P. et al. Monatshefte Für Chem.-Chem. Mon. 148, 1043-1049 (2017).
61. Bose, D. S., et al. J. Comb. Chem. 12, 100-110 (2010).
62. Albert, A. et al. J. Chem. Soc. B Phys. Org. 956(1966 ) doi: 10.1039/j29660000956.
63. Christie, G. H. et al. J. Chem. Soc. Trans. 121, 614-620 (1922).
64. Perreault, S., et al. Acc. Chem. Res. 55, 2581-2593 (2022).
65. Basilaia, M., et al. Acc. Chem. Res. 55, 2904-2919 (2022).
66. LaPlante, S. R. et al J. Med. Chem. 54, 7005-7022 (2011).
67. Toenjes, S. T. et al. Future Med. Chem. 10, 409-422 (2018).
68. Janes, M. R. et al. Cell 172, 578-589.e17 (2018).
69. Lanman, B. A., et al. Acc. Chem. Res. 55, 2892-2903 (2022).
70. Fader, L. D. et al. ACS Med. Chem. Lett. 5, 422-427 (2014).
71. Nanudorn, P. et al. Angew. Chem. n/a,.
72. Reisberg, S. H. et al. Science 367, 458-463 (2020).
73. Yao, G. et al. Nat. Commun. 13, 6488(2022 ).
74. Hegemann, J. et al. Acc. Chem. Res. 48, 1909-1919 (2015).
75. de Veer, S. J., et al. Chem. Rev. 119, 12375-12421 (2019).
76. Moore, M. J. et al. J. Am. Chem. Soc. 142, 16039-16050 (2020).
77. Bose, D. S. et al. Tetrahedron Lett. 47, 813-816 (2006).
78. Wu, J., et al, Synlett2653-2657 (2005) doi: 10.1055/s-2005-917111.
79. Muchowski, J. M. et al. Can. J. Chem. 82, 461-478 (2004).
80. De, S. K. et al Tetrahedron Lett. 46, 1647-1649 (2005).
81. Lekhok, K. C., et al. Mol. Divers. 14, 841-846 (2010).
82. Shao, Y.-D. et al Org. Lett. 21, 4831-4836 (2019).
83. Pohl, E. et al. Acta Crystallogr. D Biol. Crystallogr. 51, 48-59 (1995).
84. Weckbecker, G. et al, Nat. Rev. Drug Discov. 2, 999-1017 (2003).
85. Blankenstein, J. et al. Eur. J. Org. Chem. 2005, 1949-1964 (2005).
86. Wang, J. et al, Proc. Natl. Acad. Sci. 115, E 6404-E6410 (2018).
87. Kondo, T. et al. Chem. Commun. 57, 2416-2419 (2021).
88. Yoshisada, R., et al. Chem.—Asian J. 19, e202400336(2024 ).
89. Katoh, T., et al. RNA Biol. 15, 453-460 (2017).
90. Cui, Z., et al. Nucleic Acids Res. 46, 6387-6400 (2018).
91. Barrett, K. T., et al. Nature 509
92. Diaz, D. B., et al. Nat. Chem. 13, 218-225 (2021).
93. S̆irvinskas, M. J., et al. J. Am. Chem. Soc. 146, 24085-24093 (2024).
94. Danelius, E., et al. ACS Chem. Biol. 18, 2582-2589 (2023).
95. Shapovalov, M., et al. PLOS Comput. Biol. 15, e1006844(2019 ).
96. Rutledge, S., et al. J. Am. Chem. Soc. 125, 14336-14347 (2003).
97. Salveson, P. J. et al. Science 384, 420-428 (2024).
98. Vinogradov, A. et al. J. Am. Chem. Soc. 141, 4167-4181 (2019).
99. Kawakami, T. et al. Chem. Biol. 15, 32-42 (2008).
100. Katoh, T., et al. Cell Chem. Biol. 24, 46-54 (2017).
101. Saunders, G. J et al. Angew. Chem. Int. Ed. 61, e202206866(2022 ).
102. Li, G., et al. Org. Lett. 15, 5020-5023 (2013).
103. Fu, C. et al. J. Am. Chem. Soc. 140, 11058-11066 (2018).
104. Hantzsch, A. et al. Berichte Dtsch. Chem. Ges. 14, 1637-1638 (1881).
105. Knorr, L. et al. Berichte Dtsch. Chem. Ges. 16, 2597-2599 (1883).
106. Knorr, L. et al. Berichte Dtsch. Chem. Ges. 17, 1635-1642 (1884).
107. Bose, D. S., et al. J. Org. Chem. 68, 587-590 (2003).
108. Kennedy-Smith, J. J., et al. J. Am. Chem. Soc. 126, 4526-4527 (2004).

1. A method of peptide synthesis comprising:
contacting a translation system with a tRNA acylated with a reactive 1,3-dicarbonyl motif under conditions wherein the tRNA introduces the 1,3-dicarbonyl motif as a β- or γ-keto amide at the N-terminus of a nascent peptide, providing a substrate for Friedländer reactions for generating quinoline-peptide hybrid products.

2. The method of claim 1, further comprising converting the N-terminal keto amide into a quinoline-peptide hybrid via a Friedländer reaction with a 2-aminoarylcarbonyl substrate.

3. The method of claim 1, further comprising reacting the N-terminal keto amide with a substituted 2-aminobenzophenone to provide a Friedländer product possessing a stable biaryl atropisomeric axis.

4. The method of claim 1, wherein the peptide comprises the N-terminal keto amide and an internal 2-aminoacetophenone motif, and undergoes an intramolecular Friedländer macrocyclization reaction that embeds a quinoline pharmacophore directly within the macrocyclic backbone.

5. The method of claim 1, which effects chemo-ribosomal synthesis of atropisomeric and macrocyclic peptides with embedded heterocycles or pharmacophores, including quinolines.

6. A method of claim 1, further comprising deploying the resultant peptide carrying an N-terminal β-ketoamide as a substrate for a Friedländer reaction that converts the β-ketoamide into a heterocyclic quinoline-peptide hybrid or heterocycle-peptide hybrid.

7. A method of claim 1, further comprising use of a substituted 2-aminocarbonyl co-substrate to provide a quinoline-peptide hybrid, or other heterocycle-peptide hybrid, possessing a stable and conformationally mobile biaryl atropisomeric axis.

8. A method of claim 1, further comprising an intramolecular Friedländer reaction of the peptide carrying an N-terminal β-ketoamide with an internal 2-aminocarbonyl co-substrate or kynurenine residue, providing a substrate for an intramolecular Friedländer cyclization reaction that effectively embeds a quinoline pharmacophore into the peptide or macrocyclic backbone.
